(19) United States
(12) Patent Application Publication
(10) Pub. No.: US 2003/0060423 A1
(43) Pub. Date: Mar. 27, 2003

(54) Co-therapy for the treatment of dementia and associated behavioral manifestations comprising anticonvulsant derivatives and acetylcholinesterase inhibitors

(76) Inventor: Carlos R. Plata-Salaman, Ambler, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/229,878

(22) Filed: Aug. 28, 2002

Related U.S. Application Data
(60) Provisional application No. 60/315,978, filed on Aug. 30, 2001.

(51) Int. Cl. A61K 31/70; A61K 31/35; A61K 31/255; A61K 31/36

(52) U.S. Cl. 514/23; 514/459; 514/517; 514/464

(57) ABSTRACT

The present invention describes therapy for the treatment of dementia, memory disorders and the behavioral, psychiatric and/or psychological manifestations or symptoms associated with dementia or a memory disorder comprising co-therapy with a therapeutically effective amount of one or more acetylcholinesterase inhibitors with one or more anticonvulsant derivatives.

### CROSS REFERENCE TO RELATED APPLICATION

This application claims priority from U.S. provisional application Serial No. 60/315,978, filed Aug. 30, 2001, the contents of which are hereby incorporated by reference.

### BACKGROUND OF THE INVENTION

Alzheimer's disease (AD) is a chronic neurodegenerative disorder characterized by memory loss and dementia. As in the case of AD, non-Alzheimer's dementias are associated with memory loss and dementia. Both AD and non-AD dementias are also often accompanied by behavioral, psychiatric and/or psychological symptoms including psychosis, depression, anxiety and agitation, and other changes in mood and social withdrawal (Jost B. C., Grossberg G. T., J Am Geriatr Soc 1996, 44(9) pp1078-81). In fact, behavioral, psychiatric and/or psychological symptoms of dementia can occur in 60-90% of patients with Alzheimer's disease (AD) or other dementing illnesses, and are critically important since they are the source of significant caregiver stress and can contribute to the caregiver burnout syndrome (Rojas-Fernandez C. H., Lanctot K. L., Allen D. D., MacKnight C., Pharmacotherapy 2001 21(1) pp74-102; Jann M. W., Brandt N., J Am Pharm Assoc (Wash) 2000, 40(5 Suppl 1), S50-1; Fazio S., Bartelt T., Am Fam Physician 1999 60(7) pp2165-6).

The behavioral, psychiatric and/or psychological clinical manifestations associated with dementia or a memory disorder, more particularly with Alzheimer's disease (AD), can be assessed through clinically accepted scales, for instance, the Brief Psychiatric Rating Scale, the Alzheimer's Disease Assessment Scale-noncognitive, the Relative's Assessment of Global Symptomatology, the Dementia Behavior Disturbance Scale, the Neuropsychiatric Inventory, the Cornell Scale for Depression in Dementia, the Cohen-Mansfield Agitation Inventory, Geriatric Depression Scale, Behavior Rating Scale, Disability Assessment for Dementia, Caregiver time, and the Dementia Mood Assessment Scale (Cummings J. L. Neurology 1997 48(5 Suppl 6) S10-6; Perrault A., Oremus M., Demers L., Vida S., Wolfson C., J Geriatr Psychiatry Neurol 2000, 13(4) pp181-96).

The treatment of behavioral, psychiatric and/or psychological manifestations in patients with dementia or a memory disorder in the primary care, hospital and nursing home settings includes the use of antipsychotics, antidepressants, anxiolytics and anti-epileptics/anticonvulsants such as carbamazepine and valproic acid (Stoppe G., Staedt J., Z Gerontol Geriatr 1999 32(3) pp153-8; Schatzberg A. F., DeBattista C., J Clin Psychiatry 1999, 60 Suppl 15 pp17-20; Small G. W., Am J Med 1998 104(4A) pp32S-42S; Rojas-Fernandez C. H., Lanctot K. L., Allen D. D., MacKnight C., Pharmacotherapy 2001 21(1) pp74-102).

Compounds of Formula I:

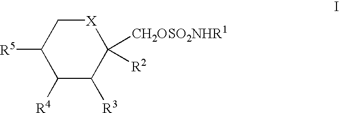

are structurally novel antiepileptic compounds that are highly effective anticonvulsants in animal tests (MARYANOFF, B. E., NORTEY, S. O., GARDOCKI, J. F., SHANK, R. P. AND DODGSON, S. P. J. Med. Chem. 1987, 30, 880-887; MARYANOFF, B. E., COSTANZO, M. J., SHANK, R. P., SCHUPSKY, J. J., ORTEGON, M. E., AND VAUGHT J. L. Bioorg. Med. Chem. Lett. 1993, 3, 2653-2656; SHANK, R. P., GARDOCKI, J. F., VAUGHT, J. L., DAVIS, C. B., SCHUPSKY, J. J., RAFFA, R. B., DODGSON, S. J., NORTEY, S. O., MARYANOFF, B. E. Epilepsia 1994, 35, 450-460; MARYANOFF, B. E., COSTANZO, M. J., NORTEY, S. O., GRECO, M. N., SHANK, R. P., SCHUPSKY, J. J., ORTEGON, M. P., VAUGHT, J. L. J. Med. Chem. 1998, 41, 1315-1343).

These compounds are covered by three US patents: U.S. Pat. No. 4,513,006, No. 5,242,942, and No. 5,384,327. One of these compounds 2,3:4,5-bis-O-(1-methylethylidene)-β-D-fructopyranose sulfamate known as topiramate has been demonstrated in clinical trials of human epilepsy to be effective as adjunctive therapy or as monotherapy in treating simple and complex partial seizures and secondarily generalized seizures (E. FAUGHT, B. J. WILDER, R. E. RAMSEY, R. A. REIFE, L D. KRAMER, G. W. PLEDGER, R. M. KARIM et. al., Epilepsia 1995, 36 (S4), 33; S. K. SACHDEO, R. C. SACHDEO, R. A. REIFE, P. LIM and G. PLEDGER, Epilepsia 1995, 36 (S4), 33; T. A. GLAUSER, Epilepsia 1999, 40 (S5), S71-80; R. C. SACHDEO, Clin. Pharmacokinet. 1998, 34, 335-346), and is currently marketed for the treatment of seizures in patients with simple and complex partial epilepsy and seizures in patients with primary or secondary generalized seizures in the United States, Europe and most other markets throughout the world.

Compounds of Formula I were initially found to possess anticonvulsant activity in the traditional maximal electroshock seizure (MES) test in mice (SHANK, R. P., GARDOCKI, J. F., VAUGHT, J. L., DAVIS, C. B., SCHUPSKY, J. J., RAFFA, R. B., DODGSON, S. J., NORTEY, S. O., and MARYANOFF, B. E., Epilepsia 1994, 35, 450-460). Subsequent studies revealed that Compounds of Formula I were also highly effective in the MES test in rats. Topiramate was also found to effectively block seizures in several rodent models of epilepsy (J. NAKAMURA, S. TAMURA, T. KANDA, A. ISHII, K. ISHIHARA, T. SERIKAWA, J. YAMADA, and M. SASA, Eur. J. Pharmacol. 1994, 254, 83-89), and in an animal model of kindled epilepsy (A. WAUQUIER and S. ZHOU, Epilepsy Res. 1996, 24, 73-77).

Shank et al., in PCT application WO98/00124 disclose the use of compound of formula I for the treatment of postischemic neurodegeneration. Further, it has been reported that topiramate has a dose- and use-dependent neuroprotective effect, when used 2 hours after MCA embolization in a rat model of focal ischemia (Yang, Y., Shuaib, A., Li, Q., Siddiqui, M. M., Brain Research, 1998, 804(2), 169-76). The neuroprotective effect of topiramate against neuronal damage following global ischemia in gerbils has also been described (Lee, S. R., Kim, S. P., Kim, J. E., Neuroscience Letters, 2000, 281(2-3), 183-6). More recently, Shank, R. P. in PCT application WO00/61138 discloses the use of compounds of formula I for the treatment of chronic neurodegenerative disorders.

There is some suggestion that topiramate may impair attention in some individuals, a frequently noted side effect of anti-epileptic medication (Burton, L. A., and Harden, C., Epilepsy Research, 1997, 27, 29-32). It has also been reported that topiramate, under certain circumstances, may have a negative impact on cognition, consistent with subjective complaints of some patients (Thompson, P. J., Baxendale, S. A., Duncan, J. S., Sander, J. W. A. S., Journal of Neurology, Neurosurgery & Psychiatry, 2000, 69(5), 636-641).

Shank, R. P. in U.S. Pat. No. 5,753,693 discloses the use of compounds of formula I for the treatment of manic-depressive bipolar disorder. van Kammen, D., WIPO publication WO00/32183 discloses the use of compounds of formula I for the treatment of schizophrenia. van Kammen, D. in WIPO publication WO00/28945 discloses the use of compounds of formula I for the treatment of post traumatic stress disorder. Berlant., J. In WIPO publication WO00/72841 discloses the use of compounds of formula I for the treatment of post traumatic stress disorder.

Acetylcholinesterase (AChE) is an enzyme that plays a pivotal role in cholinergic (acetylcholine) neurotransmission. Physiologically, the hydrolysis of acetylcholine to acetate and choline serves to inactivate acetylcholine molecules released from synaptic terminals and thereby terminate the synaptic signalling event initiated by the release of acetylcholine (ACh) from the nerve terminal. AChE inhibitors are a class of compounds which inhibit the enzyme that degrades acetylcholine. Thus, by inhibiting AChE, the residence time of acetylcholine in the synapsis is prolonged and consequently acetylcholine action is enhanced (Schneider L S, Clin Geriatr Med May 2001;17(2):337-58; Grutzendler J, Morris J C, Drugs 2001;61 (1):41-52). Acetylcholinesterase inhibitors include galantamine, rivastigmine, donepezil, tacrine, and metrifonate (Grutzendler J, Morris J C, Drugs 2001;61 (1):41-52).

Galantamine, also known as galanthamine or (4aS,6R,8aS)-4a,5,9,10,11,12-hexahydro-3-methoxy-11-methyl-6H-benzofuro[3a,3,2-ef][2]benzazepin-6-ol, is a naturally occurring organic substance, which may be derived from bulbs of the common snowdrop and several Amaryllidaceae plants. More recently, galantamine has been the subject of clinical evaluation for symptomatic treatment of neurological and behavioral signs associated with Alzheimer's Disease, and is currently approved or pending approval for marketing in many world markets under the trade name REMINYL® (SRAMEK, J. J., FRACKIEWICZ, E. J., CUTLER, N. R., Expert Opin. Invest. Drugs, 2000, 9, 2393-2402).

The known pharmacology of galantamine incudes an ability to inhibit the AChE (BORES, G. M., HUGER, F. P., PETKO, W., et. al., J. Pharmacol. Exp. Ther., 1996, 277, 728-738). The therapeutic value of an AChE-inhibitor, such as galantamine, derives from the fact that in the brain of AD patients, some of the neurons that release ACh as a synaptic signalling messenger (neurotransmitter) are dysfunctional or non-functional, due to cell death or synaptic degeneration. AChE inhibitors enhance ACh-mediated synaptic activity in this pathological circumstance by prolonging the time that ACh molecules released by the remaining functional synaptic terminals are available to activate ACh receptors in the membrane of the postsynaptic neurons.

More recently, new pharmacological properties of galantamine have been discovered which suggests that galantamine may enhance the activity of ACh by mechanisms independent of its ability to inhibit AChE such as allosteric modulation of nicotinic receptors (ABUQUERQUE, E. X., ALKONODON, M., PEREIRA, E. F. R., et.al., J. Pharmacol. Exp. Ther., 1997, 280, 1117-1136; Coyle J, Kershaw P, Biol Psychiatry Feb. 1, 2001;49(3):289-99). Galantamine has also been reported to have benefits on the behavioral and psychiatric symptoms of AD (Blesa R., Dement Geriatr Cogn Disord 2000 11 Suppl 1, pp28-34; Scott L. J., Goa K. L., Drugs 2000 60(5) pp1095-122; Coyle J, Kershaw P, Biol Psychiatry Feb. 1, 2001;49(3):289-99).

We now disclose co-therapy comprising administration of a therapeutically effective amount of one or more acetylcholinesterase inhibitors with one or more compounds of Formula I:

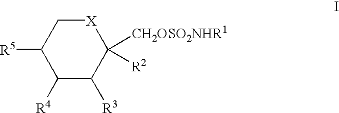

for the treatment of dementia, memory disorders and/or the behavioral, psychiatric and/or psychological manifestations or symptoms associated with dementia or a memory disorder, regardless of underlying cause, which has not yet been contemplated in the art.

### SUMMARY OF THE INVENTION

The present invention is directed to the treatment of dementia or a memory disorder with co-therapy comprising administration of a therapeutically effective amount of one or more acetylcholinesterase inhibitors with one or more compounds of the following formula I:

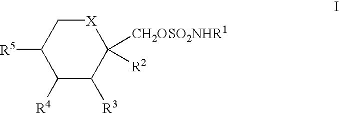

wherein X is O or CH₂, and R¹, R², R³, R⁴ and R⁵ are as defined hereinafter. Preferably, the method for treating dementia or a memory disorder comprises co-therapy with a therapeutically effective amount of galantamine with topiramate.

In one embodiment of the present invention is a method for the treatment of behavioral, psychiatric and/or psychological manifestations or symptoms of dementia or a memory disorder which comprises co-therapy with a therapeutically effect amount of one or more acetylcholinesterase inhibitors with one or more compounds of the following formula I:

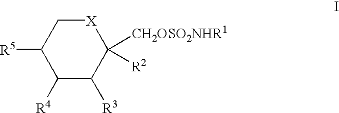

wherein X is O or CH₂, and R¹, R², R³, R⁴ and R⁵ are as defined hereinafter. Preferably, the method for treating behavioral, psychiatric and/or psychological manifestations or symptoms of dementia or a memory disorder comprises co-therapy with a therapeutically effective amount of galantamine with topiramate.

In a preferred embodiment of the present invention is a method for the treatment of dementia as a result of Alzheimer's disease which comprises co-therapy with a therapeutically effective amount of galantamine with topiramate.

In another preferred embodiment of the present invention is a method for the treatment of the behavioral, psychiatric and/or psychological manifestations or symptoms of dementia as a result of Alzheimer's disease which comprises co-therapy with a therapeutically effective amount of galantamine with topiramate.

Illustrative of the invention is a pharmaceutical composition comprising a pharmaceutically acceptable carrier, one or more compounds of formula I and one or more acetylcholinesterase inhibitors. An illustration of the invention is a pharmaceutical composition made by mixing one or more compounds of formula I and one or more acetylcholinesterase inhibitors and a pharmaceutically acceptable carrier. Illustrating the invention is a process for making a pharmaceutical composition comprising mixing one or more compounds of formula I and one or more acetylcholinesterase inhibitors and a pharmaceutically acceptable carrier. Preferably, the compound of formula I is topiramate and the acetylcholinesterase inhibitor is galantamine.

Exemplifying the invention are methods of treating dementia, a memory disorder or behavioral, psychiatric and/or psychological manifestations or symptoms associated with dementia or a memory disorder in a subject in need thereof comprising co-therapy with a therapeutically effective amount of any of the pharmaceutical compositions described above.

Another example of the invention is the use of a therapeutically effective amount of one or more compounds of formula I and one or more acetylcholinesterase inhibitors described herein in the preparation of a medicament for treating (a) dementia, (b) memory loss, (c) mental deterioration, (d) diminished mental capacity, (e) loss of cognition or (f) the behavioral, psychiatric and/or psychological manifestations or symptoms of dementia or a memory disorder, in a subject in need thereof. Preferably, the compound of formula I is topiramate and the acetylcholinesterase inhibitor is galantamine.

### DETAILED DESCRIPTION OF THE INVENTION

As used herein, the term “dementia” shall include the deterioration of intellectual and other mental processes, regardless of underlying cause that impairs daily activities and is the result of a deficit in a previously successful performance. Suitable examples of dementia include, but are not limited to, dementia as a result of Alzheimer's disease, vascular related dementia, multi-infarct related dementia, dementia as a result of head trauma, dementia as a result of diffuse brain damage, dementia pugilistica, dementia as a result of Huntington's disease, dementia as a result of alcoholism, dementia as a result of diffuse white matter disease, dementia associated with Parkinson's disease, dementia as a result of Lewy body disease, dementia as a result of Pick's disease, dementia as a result of multisystem degeneration, dementia as a result of progressive supranuclear palsy, dementia associated with the ALS-Parkinson's-Dementia complex of Guam, frontal lobe dementia, and dementia as a result of cortical basal degeneration.

As used herein, the term “memory disorder” shall include memory loss, mental deterioration, diminished mental capacity and loss of cognition.

As used herein, the term “behavioral, psychiatric and/or psychological manifestations or symptoms associated with dementia or a memory disorder” shall include psychoses, depression, anxiety, agitation, irritability, changes in mood, social withdrawal, language difficulties, impaired comprehension and disorientation associated with dementia or a memory disorder, regardless of underlying cause.

As used herein, the term “improvement” shall mean a positive result to treatment. More particularly, “improvement” or a “positive result” shall mean that one or more parameters used to measure the progression of a disease or disorder are (a) unchanged relative to a baseline value (while a decrease relative to baseline occurs in untreated patients or patients treated with placebo), (b) improved relative to a baseline value or (c) decreased relative to a baseline value, but where the decrease is by an amount which is less than that which would be expected if no treatment were administered or alternatively the decrease is by an amount which is less than that observed for patients treated with placebo, as in a placebo controlled clinical trial.

As used herein, the term “subject” refers to an animal, preferably a mammal, most preferably a human, who is the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. More particularly, in the present invention directed to co-therapy comprising administration of one or more compound(s) of formula I and one or more acetylcholinesterase inhibitor(s), “therapeutically effective amount” shall mean that amount of the combination of agents taken together so that the combined effect elicits the desired biological or medicinal response. For example, the therapeutically effective amount of co-therapy comprising administration of a compound of formula I and an acetylcholinesterase inhibitor would be the amount of the compound of formula I and the amount of the acetylcholinesterase inhibitor that when taken together or sequentially have a combined effect that is therapeutically effective. Further, it will be recognized by one skilled in the art that in the case of co-therapy with a therapeutically effective amount, as in the example above, the amount of the compound of formula I and/or the amount of the acetylcholinesterase inhibitor individually may or may not be therapeutically effective.

As used herein, the term “co-therapy” shall mean treatment of a subject in need thereof by administering one or more compounds of formula I with one or more acetylcholinesterase inhibitors, wherein the compound(s) of formula I and the acetylcholinesterase inhibitor(s) are administered by any suitable means, simultaneously, sequentially, separately or in a single pharmaceutical formulation. Where the compound(s) of formula I and the acetylcholinesterase inhibitor(s) are administered in separate dosage forms, the number of dosages administered per day for each compound may be the same or different. The compound(s) of formula I and the acetylcholinesterase inhibitor(s) may be administered via the same or different routes of administration. Examples of suitable methods of administration include, but are not limited to, oral, intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula I and the acetylcholinesterase inhibitor(s) may be administered according to simultaneous or alternating regimens, at the same or different times during the course of the therapy, concurrently in divided or single forms.

Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

As used herein, the term “composition” is intended to encompass a product comprising the specified ingredients in the specified amounts, as well as any product which results, directly or indirectly, from combinations of the specified ingredients in the specified amounts.

As used herein, the term “synergy” or “synergistic effect” when used in connection with a description of the efficacy of a combination of agents, shall mean any measured effect of the combination which is greater than the value predicted from a sum of the effects of the individual agents. (Greco, W. R., Bravo, G., Parsons, J. C., Pharmacol Rev, 1995, 47, 331-385).

The compounds of formula I of the present invention are compounds of the following general formula I:

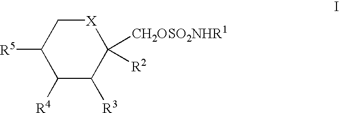

wherein

X is CH₂ or oxygen;

R¹ is hydrogen or alkyl; and

R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula II:

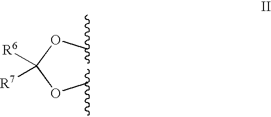

wherein

R⁶ and R⁷ are the same or different and are hydrogen, lower alkyl or are alkyl and are joined to form a cyclopentyl or cyclohexyl ring.

R¹ in particular is hydrogen or alkyl of about 1 to 4 carbons, such as methyl, ethyl and iso-propyl. Alkyl throughout this specification includes straight and branched chain alkyl. Alkyl groups for R², R³, R⁴, R⁵, R⁶ and R⁷ are of about 1 to 3 carbons and include methyl, ethyl, iso-propyl and n-propyl. When X is CH₂, R⁴ and R⁵ may combine to form a benzene ring fused to the 6-membered X-containing ring, i.e., R⁴ and R⁵ are defined by the alkatrienyl group ═C—CH═CH—CH═.

A particular group of compounds of formula I is that wherein X is oxygen and both R² and R³ and R⁴ and R⁵ together are methylenedioxy groups of the formula II, wherein R⁶ and R⁷ are both hydrogen, both alkyl or combine to form a spiro cyclopentyl or cyclohexyl ring, in particular where R⁶ and R⁷ are both alkyl such as methyl. A second group of compounds is that wherein X is CH₂ and R⁴ and R⁵ are joined to form a benzene ring. A third group of compounds of formula I is that wherein both R² and R³ are hydrogen.

The compounds of formula I may be synthesized by the following methods:

(a) Reaction of an alcohol of the formula RCH₂OH with a chlorosulfamate of the formula ClSO₂NH₂ or ClSO₂NHR¹ in the presence of a base such as potassium t-butoxide or sodium hydride at a temperature of about −20° to 25° C. and in a solvent such as toluene, THF, or dimethylformamide wherein R is a moiety of the following formula III:

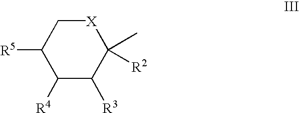

(b) Reaction of an alcohol of the formula RCH₂OH with sulfurylchloride of the formula SO₂Cl₂ in the presence of a base such as triethylamine or pyridine at a temperature of about −40° to 25° C. in a solvent such as diethyl ether or methylene chloride to produce a chlorosulfate of the formula RCH₂OSO₂Cl.

The chlorosulfate of the formula RCH₂OSO₂Cl may then be reacted with an amine of the formula R¹NH² at a temperature of abut 40° to 25° C. in a solvent such as methylene chloride or acetonitrile to produce a compound of formula I. The reaction conditions for (b) are also described by T. Tsuchiya et al. in Tetrahedron Lett., 1978, 3365.

(c) Reaction of the chlorosulfate RCH₂OSO₂Cl with a metal azide such as sodium azide in a solvent such as methylene chloride or acetonitrile yields an azidosulfate of the formula RCH₂OSO₂N₃ as described by M. Hedayatullah in Tetrahedron Lett. 1975, 2455. The azidosulfate is then reduced to a compound of formula I wherein R¹ is hydrogen by catalytic hydrogenation, e.g. with a noble metal and H₂ or by heating with copper metal in a solvent such as methanol.

The starting materials of the formula RCH₂OH may be obtained commercially or as known in the art. For example, starting materials of the formula RCH₂OH wherein both R² and R³ and R⁴ and R⁵ are identical and are of the formula II may be obtained by the method of R. F. Brady in Carbohydr. Res. 1970, 14, 35 or by reaction of the trimethylsilyl enol ether of a R⁶COR⁷ ketone or aldehyde with fructose at a temperature of about 25° C., in a solvent such a halocarbon, e.g. methylene chloride in the presence of a protic acid such as hydrochloric acid or a Lewis Acid such as zinc chloride. The trimethylsilyl enol ether reaction is described by G. L. Larson et al. in J. Org. Chem. 1973, 38, 3935.

Further, carboxylic acids and aldehydes of the formulae RCOOH and RCHO may be reduced to compounds of the formula RCH₂OH by standard reduction techniques, e.g. reaction with lithium aluminum hydride, sodium borohydride or borane-THF complex in an inert solvent such a diglyme, THF or toluene at a temperature of about 0° to 100° C., e.g. as described by H. O. House in “Modern Synthetic Reactions”, 2nd Ed., pages 45 to 144 (1972).

The compounds of formula I may also be made by the processes disclosed in U.S. Pat. No. 4,513,006, No. 5,242,942, and No. 5,384,327, which are incorporated by reference herein.

The compounds of formula I include the various individual isomers as well as the racemates thereof, e.g., the various alpha and beta attachments, i.e., below and above the plane of the drawing, of R², R³, R⁴ and R⁵ on the 6-membered ring. Preferably, the oxygen of the methylenedioxy group of formula II are attached on the same side of the 6-membered ring.

Acetylcholinesterase inhibitors include galantamine, rivastigmine, donepezil, tacrine and metrifonate.

Galantamine, also known by the brand name Reminyl®, is known chemically as (4aS,6R,8aS)-4a,5,9,10,11,12-hexahydro-3-methoxy-11-methyl-6H-benzofuro[3a,3,2-ef][2]benzazepin-6-ol. Galantamine is a natural substance, which can be derived from bulbs of the common snowdrop and several Amaryllidaceae plants by a solvent extraction process (Sramek J J, Frackiewicz E J, Cutler N R. Exp Opin Investig Drugs 2000, 9:2393-2402). Galantamine is approved in the U.S. for the treatment of mild to moderate AD, with recommended dosage levels of 8 to 32 mg/day, preferably 16 to 32 mg/day.

Rivastigmine, also known by the brand name Exelon®, is known chemically as (S)-N-ethyl-N-methyl-3-[1-(dimethylamino)ethyl]-phenyl carbamate hydrogen-(2R,3R)-tartrate. Rivastigmine is approved in the U.S. for the treatment of mild to moderate dementia of the Alzheimer's type, with recommended dosage levels of 6 to 16 mg/day.

Donepezil, also known by the brand name Aricept®, is known chemically as (±)-2,3-dihydro-5,6-dimethoxy-2-[[1-(phenylmethyl)-4-piperidinyl]methyl]-1H -inden-1-one hydrochloride. Donepezil is approved in the U.S. for the treatment of mild to moderate dementia of the Alzheimer's type, with recommended dosage levels of 5 to 10 mg/day, preferably 5 mg/day.

Tacrine, also known by the brand name Cognex®, is known chemically as 1,2,3,4-tetrahydro-9-acridinamine. Tacrine is approved in the U.S. for the treatment of mild to moderate dementia of the Alzheimer's type, with recommended dosage levels of 80 to 160 mg/day, preferably 120 mg/day.

Metrifonate, is known chemically as (2,2,2-trichloro-1-hydroxyethyl)-dimethyl ester (6Cl, 8Cl, 9Cl) phosphonic acid. Metrifonate is currently being evaluated in clinical studies for effectiveness in the treatment of mild to moderate Alzheimer's disease with a loading dose of 0.5 mg/kg to 2.5 mg/kg and a maintenance dose of 0.2 mg/kg to 0.65 mg/kg daily.

The effectiveness of co-therapy comprising administration of a therapeutically effective amount of one or more acetylcholinesterase inhibitors and one or more compounds of formula I to treat dementia, a memory disorder or the behavioral, psychiatric and/or psychological manifestations or symptoms of dementia or a memory disorder may be determined according to a case study as described in more detail herein.

### EXAMPLE 1

A patient presents to a physician complaining of some or all of the following clinical manifestations and/or a relative or caregiver reports similar manifestations: memory loss, language difficulties, impaired comprehension, difficulties with problem solving (which were not present before), increased need for reminders, disorientation, periods of anxiety, irritability, agitation, depression, withdrawal and/or psychosis, and/or a decrease in activities of daily living.

The patient is assessed for the extent of behavioral, psychiatric and psychological manifestations of Alzheimer's disease and changes in cognition, function and quality of life using assessment scales, including but not limited to, the Brief Psychiatric Rating Scale, the Alzheimer's Disease Assessment Scale cognitive and noncognitive, the Clinical Dementia Rating, the Global Deterioration Scale, the Mini-Mental State Examination, the Dementia of the Alzheimer Type Inventory, the Brief Cognitive Rating Scale, the Blessed Dementia Scale, the Cognitive Capacity Screening Examination, the Cognitive Levels Scale, the Clinical Dementia Rating, the Relative's Assessment of Global Symptomatology, the Dementia Behavior Disturbance Scale, the Neuropsychiatric Inventory, the Cornell Scale for Depression in Dementia, the Cohen-Mansfield Agitation Inventory, Geriatric Depression Scale, Behavior Rating Scale, Disability Assessment for Dementia, Caregiver time, the Dementia Mood Assessment Scale, the Katz Index of Activities of Daily Living, and the Lawton Instrumental Activities of Daily Living Scale (Bryant J, Clegg A, Nicholson T, McIntyre L, De Broe S, Gerard K, Waugh N., Health Technol Assess 2001 5(1):1-137; Cummings J. L. Neurology 1997 48(5 Suppl 6) S10-6; Demers L, Oremus M, Perrault A, Wolfson C., J Geriatr Psychiatry Neurol 2000 13(4):161-169; Perrault A., Oremus M., Demers L., Vida S., Wolfson C., J Geriatr Psychiatry Neurol 2000, 13(4) pp181-96).

The patient is started on a treatment protocol of topiramate and galantamine. Topiramate is prescribed in the range of about 10 to 650 mg/day; galantamine is prescribed in the range of about 4 to 32 mg/day. Topiramate and galantamine are administered to the patient according to defined titration and dosing regimens as those described in their respective package inserts.

After 3 months, 6 months and again at 12 months, the patient is re-evaluated on the assessment scales and shows an improvement in cognition and/or an improvement in behavioral, psychiatric and/or psychological manifestations or symptoms, and/or improvement in the activities of daily living, and/or reduction in caregiver burden. For example, the patient exhibits one or more of the following improvements: the patient is less anxious, less irritable, less agitated, less depressed, less apathic, the patient's disorientation is less frequent or of shorter duration, the patient experiences fewer or less intense period of psychosis, is more socially active (shows less social withdrawal), exhibits improved comprehension and/or improved language skills and/or more stable moods (e.g. exhibit less frequent or rapid mood swings), exhibits fewer decreases in instrumental activities of daily living and/or less decline in self-care.

The patient is judged as having a positive result or improvement if the patient indicates or exhibits improved cognition, improved memory, decreased mental deterioration, improved mental capacity or an improvement in behavioral, psychiatric and/or psychological manifestations or symptoms. More particularly, the treatment is judged effective (i.e. the patient shows a positive result or improvement) if one or more parameters used to measure cognition, memory loss, mental deterioration, mental capacity or the behavioral, psychiatric and/or psychological manifestations or symptoms are (a) unchanged relative to a baseline value (while a decrease relative to baseline is expected to occur in an untreated patient/if the patient were untreated), (b) improved relative to a baseline value or (c) decreased relative to a baseline value, but where the decrease is by an amount which is less than that which would be expected if no treatment were administered.

Thus, for treating dementia, a memory disorder or the behavioral, psychiatric and/or psychological manifestations or symptoms of dementia or a memory disorder, one or more compounds of formula I may be administered as co-therapy with one or more acetylcholinesterase inhibitors. Preferably, the co-therapy comprises administration of a therapeutically effective amount of galantamine with topiramate.

Preferably, the co-therapy comprising administration of the compound of formula I and an acetylcholinesterase inhibitor produces a synergistic effect.

Wherein the compound of formula I is topiramate, the topiramate is preferably administered in an amount in the range of about 10 to about 650 mg daily, more preferably in an amount in the range of about 25 to about 325 mg once ot twice daily. Topiramate, is currently available in unit dosage forms of 15 mg, 25 mg, 100 mg and 200 mg.

Wherein the acetylcholinesterase inhibitor is galantamine, the galantamine is administered in an amount in the range of about 2 to about 32 mg daily, more preferably in an amount in the range of about 4 to about 24 mg once or twice daily. Galantamine, is currently available under the brand name Reminyl®, in unit dosage forms (tablets) of 4 mg, 8 mg and 12 mg.

To prepare the pharmaceutical compositions of the present invention, one or more compounds of the formula I, one or more acetylcholinesterase inhibitors or a combination thereof, are intimately admixed with a pharmaceutically acceptable carrier according to conventional pharmaceutical compounding techniques, wherein the carrier may take a wide variety of forms depending on the form of the preparation desired for administration, e.g. i.v. sterile injectable formulations will be prepared using appropriate solubilizing agents.

Preferably the pharmaceutical composition(s) are in unit dosage forms such as tablets, pills, caplets, capsules (each including immediate release, timed release and sustained release formulations), powders, granules, sterile parenteral solutions or suspensions, metered aerosol or liquid sprays, drops, ampoules, autoinjector devices or suppositories; for oral, parenteral, intranasal, sublingual or rectal administration, or for administration by inhalation or insufflation.

For preparing solid compositions such as tablets, the principal active ingredients are mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compounds of the present invention, or a pharmaceutically acceptable salt thereof. When referring to these preformulation compositions as homogeneous, it is meant that the active ingredients are dispersed evenly throughout the composition so that the composition may be readily subdivided into equally effective dosage forms such as tablets, pills and capsules. This solid preformulation composition is then subdivided into unit dosage forms of the type described above containing from 5 to about 1000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

For oral administration in the form of a tablet or capsule, the active drug components may be combined with an oral, non-toxic pharmaceutically acceptable inert carrier such as ethanol, glycerol, water and the like. Moreover, when desired or necessary, suitable binders, lubricants, suspending agents, disintegrating agents, coloring agents, flavorants, sweeteners, preservatives, dyes, coatings and other inert pharmaceutical excipients can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For preparing liquid formulations, for administration orally or by injection, the principal active ingredients are mixed with a pharmaceutical carrier, e.g. aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin. Forms useful for parenteral administration include sterile solutions, emulsions and suspensions. Isotonic preparations which generally contain suitable preservatives are employed when intravenous administration is desired.

Furthermore, the active drug components may alternatively be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

The active drug components may alternatively still be administered in the form of liposome delivery systems, such as small unilamellar vesicles, large unilamellar vesicles, and multilamellar vesicles. Liposomes can be formed from a variety of phospholipids, such as cholesterol, stearylamine or phophatidylcholines.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method for treating dementia or a memory disorder in a subject in need thereof comprising co-therapy with a therapeutically effective amount of an acetylcholinesterase inhibitor and a compound of the formula I: 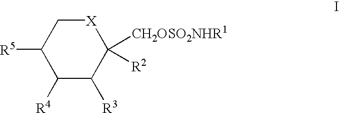
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula II: 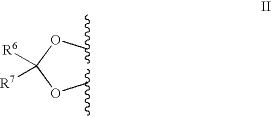
wherein
R⁶ and R⁷ are the same or different and are hydrogen, lower alkyl or are alkyl and are joined to form a cyclopentyl or cyclohexyl ring.

2. The method of claim 1 wherein the compound of formula I is topiramate.

3. The method of claim 2, wherein the amount of topiramate is from about 10 to about 650 mg daily.

4. The method of claim 3, wherein the amount of topiramate is from about 25 to about 325 mg once or twice daily.

5. The method of claim 1, wherein the acetylcholinesterase inhibitor is selected from the group consisting of galantamine, rivastigmine, donepezil, tacrine and metrifonate.

6. The method of claim 5, wherein the acetylcholinesterase inhibitor is galantamine.

7. The method of claim 6, wherein the amount of galantamine is from about 2 to about 32 mg daily.

8. The method of claim 7, wherein the amount of galantamine is from about 4 to about 24 mg daily.

9. The method of claim 1, wherein the acetylcholinesterase inhibitor is galantamine and the compound of formula I is topiramate.

10. The method of claim 1, wherein the dementia is selected from the group consisting of dementia as a result of Alzheimer's disease, vascular related dementia, multi-infarct related dementia, dementia as a result of head trauma, dementia as a result of diffuse brain damage, dementia pugilistica, dementia as a result of Huntington's disease, dementia as a result of alcoholism, dementia as a result of diffuse white matter disease, dementia associated with Parkinson's disease, dementia as a result of Lewy body disease, dementia as a result of Pick's disease, dementia as a result of multisystem degeneration, dementia as a result of progressive supranuclear palsy, dementia associated with the ALS-Parkinson's-Dementia complex of Guam, frontal lobe dementia, and dementia as a result of cortical basal degeneration.

11. The method of claim 10, wherein the dementia is dementia as a result of Alzheimer's disease.

12. The method of claim 1, wherein the memory disorder is selected from the group consisting of memory loss, diminished mental capacity, mental deterioration and loss of cognition.

13. A method for treating behavioral, psychiatric or psychological manifestations or symptoms of dementia or a memory disorder in a subject in need thereof comprising co-therapy with a therapeutically effective amount of an acetylcholinesterase inhibitor and a compound of the formula I: 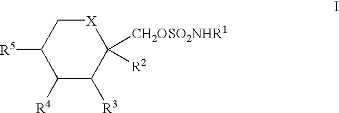
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula II: 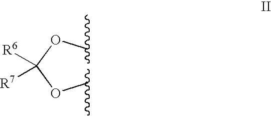
wherein
R⁶ and R⁷ are the same or different and are hydrogen, lower alkyl or are alkyl and are joined to form a cyclopentyl or cyclohexyl ring.

14. The method of claim 13 wherein the compound of formula I is topiramate.

15. The method of claim 14, wherein the amount of topiramate is from about 10 to about 650 mg daily.

16. The method of claim 15, wherein the amount of topiramate is of from about 25 to about 325 mg once or twice daily.

17. The method of claim 13, wherein the acetylcholinesterase inhibitor is selected from the group consisting of galantamine, rivastigmine, donepezil, tacrine and metrifonate.

18. The method of claim 17, wherein the acetylcholinesterase inhibitor is galantamine.

19. The method of claim 18, wherein the amount of galantamine is from about 2 to about 32 mg daily.

20. The method of claim 19, wherein the amount of galantamine is from about 4 to about 32 mg daily.

21. The method of claim 13, wherein the acetylcholinesterase inhibitor is galantamine and the compound of formula I is topiramate.

22. A pharmaceutical composition comprising galantamine, topiramate and a pharmaceutically acceptable carrier.

23. A pharmaceutical composition made by mixing galantamine, topiramate and a pharmaceutically acceptable carrier.

24. A process for making a pharmaceutical composition comprising mixing galantamine, topiramate and a pharmaceutically acceptable carrier.

26. The use of a therapeutically effective amount of topiramate and galantamine in the preparation of a medicament for treating (a) dementia, (b) memory loss, (c) mental deterioration, (d) diminished mental capacity, (e) loss of cognition or (f) behavioral, psychiatric and/or psychological manifestations or symptoms of dementia or a memory disorder in a subject in need thereof.
